(19) United States
(12) Patent Application Publication
(10) Pub. No.: US 2024/0016661 A1
(43) Pub. Date: Jan. 18, 2024

(54) VITREOUS FLOATER TREATMENT USING RESONANT SCANNER-BASED SLO

(71) Applicant: Alcon Inc., Fribourg (CH)

(72) Inventor: Zsolt Bor, San Clemente, CA (US)

(21) Appl. No.: 18/351,401

(22) Filed: Jul. 12, 2023

Related U.S. Application Data
(60) Provisional application No. 63/388,911, filed on Jul. 13, 2022.

(51) Int. Cl. A61F 9/008

(52) U.S. Cl. CPC A61F 9/00814; A61F 2009/00885; A61F 2009/00874; A61F 2009/00897

(57) ABSTRACT

Particular embodiments disclosed herein provide a system for treating vitreous floaters. Light from a first laser (e.g., laser diode) is focused at a plurality of points within a vitreous of a patient's eye using a scanner while measuring reflected light from the plurality of points. The reflected light (e.g., images) are evaluated to identify a portion of the plurality of points corresponding to one or more vitreous floaters. Second light from a second laser (e.g., pulsed laser) is focused at the portion of the plurality of points using the scanner in order to disintegrate the one or more vitreous floaters.

### CROSS-REFERENCE TO RELATED APPLICATIONS

This application claims benefit of and priority to U.S. Provisional Patent Application No. 63/388,911, filed Jul. 13, 2022, the entire contents of which are incorporated by reference herein in its entirety.

### BACKGROUND

Light received by the human eye, passes through the transparent cornea covering the iris and pupil of the eye. The light is transmitted through the pupil and is focused by a crystalline lens positioned behind the pupil in a structure called the capsular bag. The light is focused by the lens and the cornea onto the retina, which includes rods and cones capable of generating nerve impulses in response to the light. The space between the lens and the retina is occupied by a clear gel known as the vitreous.

Through various causes, floaters may be present in the vitreous. A floater is typically formed of a clump of cells, collagen fibers and/or other tissues and is more opaque than the surrounding vitreous. Floaters cast shadows onto the retina that cause visual disturbance for a patient, which can be quite severe in some patients.

### BRIEF SUMMARY

The present disclosure relates generally to a system for treating vitreous floaters.

Particular embodiments disclosed herein provide a method and corresponding apparatus, the method including focusing first light from a first laser at a plurality of points within a vitreous of a patient's eye using a scanner system while measuring reflected light from the plurality of points. The method includes determining, by a computer system, that the reflected light from a portion of the plurality of points corresponds to one or more vitreous floaters. In response to determining that the reflected light from the portion of the plurality of points corresponds to one or more vitreous floaters, second light from a second laser is focused at the portion of the plurality of points using the scanner system in order to disintegrate the one or more vitreous floaters.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 is a schematic cross-sectional representation of an eye having a floater.

FIG. 2 is a schematic diagram of a scanning laser ophthalmoscope (SLO) and treatment laser system for treating vitreous floaters, in accordance with certain embodiments.

FIG. 3 is an SLO image of a vitreous floater, in accordance with certain embodiments.

FIG. 4 is a diagram showing laser scanning paths of an SLO, in accordance with certain embodiments.

FIG. 5 is a diagram showing a boundary containing a floater, in accordance with certain embodiments.

FIG. 6 is a diagram illustrating a scanning and activation pattern of a treatment laser, in accordance with certain embodiments.

FIG. 7 is a diagram illustrating cavitation bubbles from activation of a treatment laser, in accordance with certain embodiments.

FIG. 8 illustrates the implementation of a scanning path from a sinusoidal path, in accordance with certain embodiments.

FIG. 9 illustrates an example computing device that implements, at least partly, one or more functionalities of an SLO and treatment laser system, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

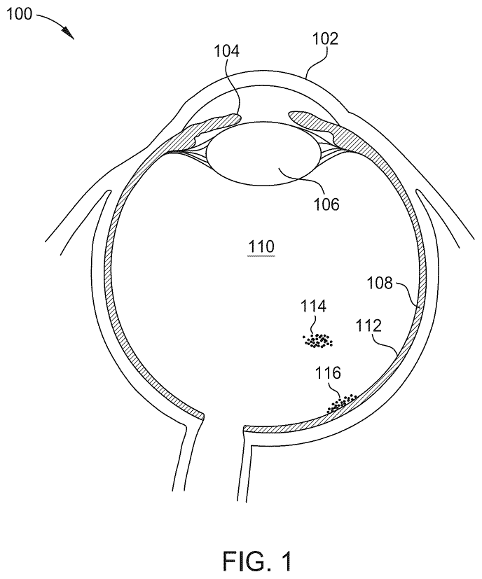

Referring to FIG. 1, a human eye 100 includes the cornea 102, which is a curved transparent layer through which light enters the eye 100. The light then passes through the anterior chamber 138, pupil 104, and lens 106 of the eye 100, respectively. The remaining volume of the globe 108 of the eye 100, known as the posterior or vitreous chamber, is filled by a clear gel known as the vitreous 110. The light is focused by the cornea 102 and lens 106 onto the retina 112 at the back of the eye 100 through the vitreous 110.

Vitreous floaters 114 are clumps of cells, collagen fibers, or other contaminants. When present, a vitreous floater 114 will cast a shadow 116 onto the retina 112. The shadow 116 sometimes may occupy a large angular extent of the field of vision of the eye 100. When sufficiently large, opaque, and/or numerous, floaters 114 can significantly reduce a patient's vision.

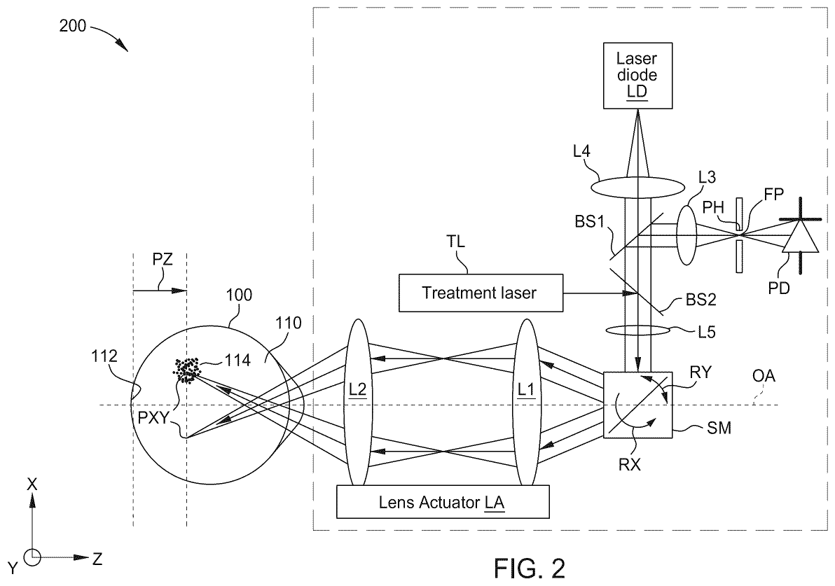

FIG. 2 illustrates an example scanning laser ophthalmoscope (SLO) and treatment laser system 200 (hereinafter “the system 200”) that may be used to implement the methods described herein. The system 200 includes a laser diode LD and a treatment laser TL. The laser diode LD is an infrared laser diode suitable for use in an SLO as known in the art. The infrared light from the laser diode LD is not visible to the patient. The treatment laser TL is a pulsed laser that can generate pulses at a high repetition rate, such as between 1 and 2 MHz, for disintegrating vitreous floaters 114. The treatment laser TL may be capable of a higher pulse repetition rate but be gated or selectively turned on to achieve this repetition rate. In some implementations, the treatment laser TL is a chirped pulse regenerative amplifier based (CPA) femtosecond treatment laser, which has a repetition rate on the order of one or more MHz. Such lasers can also operate in a “pulse per demand” operating mode, which means that, from the MHz repetition rate pulse train, pulses can be selected up to a similarly high rate. For example, pulses may be selected for output at a rate of between 1 kHz and 1 MHz, which is suitable for the methods disclosed herein. In some implementations, pulses are selected using an acousto-optic deflector, Pockels cell, or other type of pulse picking devices.

The beam from the laser diode LD is made substantially (e.g., within 1 degree of) parallel using lens L4. The beam from the laser diode LD is incident on a scanning mirror SM that rotates about at least two rotational directions. For example, the scanning mirror SM may rotate in rotational directions RX and RY, which may be defined as rotation about the X and Y axes, respectively. In some implementations, the scanning mirror SM is implemented by a first mirror rotating about rotational direction RX (“the RX mirror”) and a second mirror rotating about direction RY (“the RY mirror”). For example, the RX mirror may be implemented as a resonant scanner whereas the RY mirror is implemented as a relatively slower galvo mirror.

Light reflected from the scanning mirror SM is directed through one or more lenses L1, L2. One or both of the lenses L1, L2 may be mounted to a lens actuator LA. The lens actuator LA actuates the positions of one or both lenses L1 and L2 along the optical axis OA of the lenses L1 and L2 to change a position PZ of the focal points of the laser diode LD and the treatment laser TL along the Z axis. The Z axis may be substantially parallel (e.g., within 0.1 degrees) to the optical axis OA. The adjustment of the position PZ may be accompanied with changing the position PXY of the focal points in the XY plane using the scanning mirror SM in order to target any three-dimensional coordinate within the vitreous of the eye. In some implementations, the lenses L1 and L2 and lens actuator LA may be replaced with one or more electronically controlled optofluidic lenses that can achieve the same degree of adjustment without the use of a mechanical actuator. In some embodiments, an electrically tunable lens L5 can be added between beams splitter BS2 and scanning mirrors SM. The activation of lens L5 can move the focal point PXY to the required depth PZ. As an example, to facilitate understanding of the order of magnitude, for an average emmetropic eye, the addition of 0.36 diopter moves the focal point depth PZ by about 1 mm away from the retina. To have proper optical resolution, L1 and L2 may each be either a single lens or a compound lens system.

A portion of the light from the focal point PXY of the laser diode LD that is reflected back from the vitreous passes back through the lenses L2, L1 and is descanned by the scanning mirror SM onto a beam splitter BS1. The beam splitter BS1 directs at least a portion of the descanned light onto a photodiode PD. As is apparent from FIG. 2, light emitted from the laser diode LD is incident on the beam splitter BS1 and a portion passes therethrough to reach the vitreous 110.

In some implementations, to reduce the detection of light reflected from the cornea, lens, or other structures located somewhere other than at the focal point of the laser diode LD, a lens L3 is positioned between the beam splitter BS1 and the photodiode PD. A pinhole PH is positioned between the lens L3 and the photodiode PD and is aligned with a focal point FP of the descanned light to implement a confocal pinhole filter PH. The focal point FP is the focal point of the optical path from a focal point of the laser diode (position PXY, PZ) in the vitreous 110, which includes the effect of the lenses L1, L2, and L3. Accordingly, light reflected from any location other than position PXY, PZ will not be focused upon arrival at the pinhole PH and will be substantially blocked by the pinhole PH. The diameter of the pinhole PH may be selected to block the undesired reflections while still permitting sufficient light to pass through to be detected by the photodiode PD. To have efficient depth selection, the diameter of the pinhole PH should be about (e.g., within 10 percent of) the diffraction limited spot diameter of the lens L3. The focal point depth PZ and the pinhole PH are optically conjugated and the pinhole PH acts as a confocal filter suppressing any light that did not originate from the focal point PXY, PZ of the system 200. The lens L3 may be implemented as a single lens or as a compound lens system. Therefore, system 200 can measure the reflectivity of the retina in 2D or the reflectivity of the floater in 3D.

Various elements may include combining optics for routing light from the treatment laser TL to be precisely (e.g., within 0.001 degrees) parallel and collinear (e.g., within 0.01 μm) with light from the laser diode LD. For example, a second beam splitter BS2 may be used to redirect a portion of the light from the treatment laser TL to be parallel to and collinear with the light from the laser diode LD that passes through the second beam splitter BS2. In the illustrated implementation, the beam splitter BS2 is positioned between beam splitter BS1 and the scanning mirror SM.

The illustrated system 200 has the advantage that the light from the treatment leaser TL and light from the laser diode LD are focused exactly (e.g., within 0.01 μm) onto the same position PXY, PZ regardless of refraction due to the cornea 102 and lens 106 of the patient's eye. Accordingly, if light is scattered by a point on a floater 114 for a given state of the lenses L1, L2 and the scanning mirror SM, light from the treatment laser can be transmitted through the system 200 having the same state of the lenses L1, L2 and the scanning mirror SM in order to destroy that point of the floater 114. Accordingly, if an image of a floater is detected by the system 200 and possibly displayed on a screen of an operator, the treatment laser is automatically focused onto the floater. There is no need for co-calibration of the focus of the laser diode LD beam and the focus of the treatment laser TL beam. The lenses L1, L2 and L5 (if applicable) are selected such that the light diverges significantly after passing through position PXY, PZ in order to reduce the intensity of light incident on the retina 112. The numerical aperture of the treatment laser TL beam and of the laser diode LD beam is limited only by the pupil diameter.

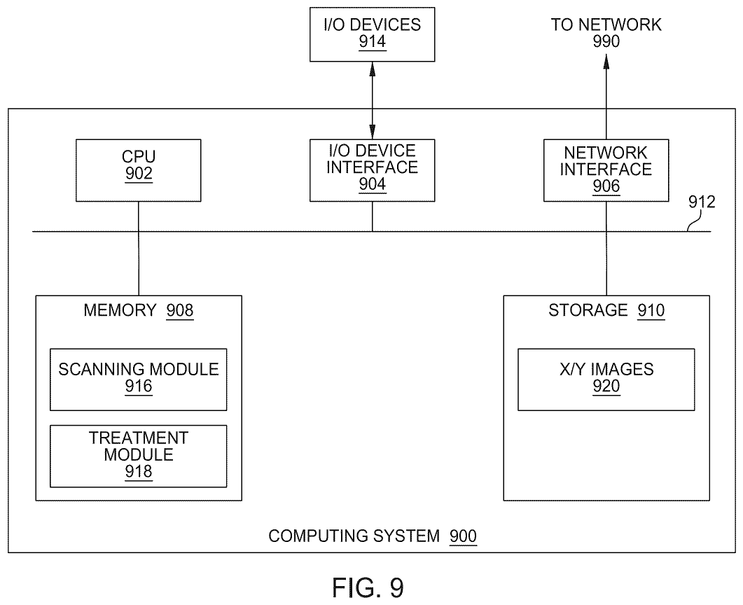

The system 200 may be coupled to a computer system, such as computer system having some or all of the attributes of the computing system 900 described below. The computer system may continuously receive the output of the photodiode PD and the information about the angular orientation of the mirrors from the encoder of RX and RY mirrors. The depth PZ position of the focus is provided by the encoder of the lens actuator LA. Combining these data, a computing system 900 (see FIG. 9) can continuously create a three-dimensional map of the vitreous and of the floater 114. The computer also can display the enface 2D or a 3D image of the floater in the form of a video.

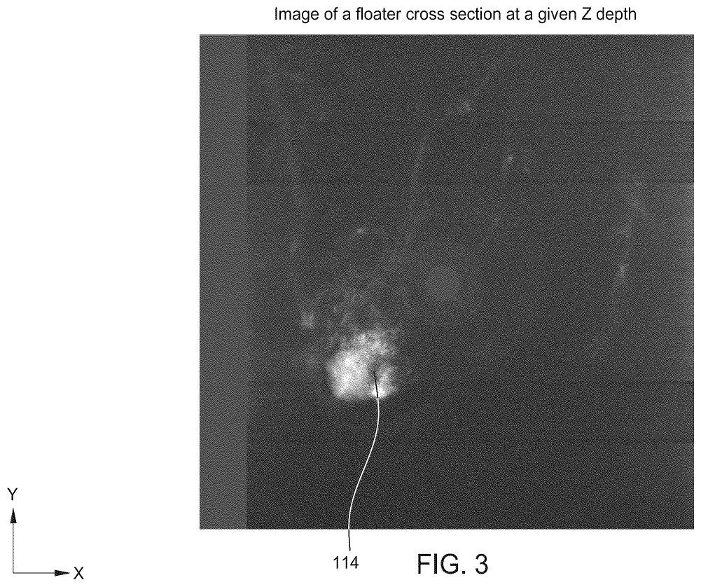

FIG. 3 illustrates an example image of a floater 114 that may be obtained using the system 200. The illustrated image may comprise a two-dimensional array of intensity values measured using the photodiode PD for a single depth PZ, i.e., an enface X/Y image, of the floater 114 embedded into the vitreous 110. The image of FIG. 3 is therefore an image of the cross section of the floater 114 at a given depth PZ. In conventional ophthalmology practice, a SLO typically is used to image the retina 112. In such cases, the focal point PXY, PZ is on the retina, i.e., PZ=0, and the SLO measures the reflectivity of laser diode light from the retina. In case of the presence of a floater 114, the shadow 116 casted by the floater appears as a darker blob on the image of the retina 112. A conventional SLO is capable of capturing images of a retina, including shadows 116 cast by floaters 114 onto the retina. The system 200, however, may have a focal plane shifted into the vitreous and away from the retina 112 to enable direct detection of light reflected from floaters 114. The enface image of a floater 114 shown in FIG. 3 is one frame of video taken at 4 frames per second. The video was taken on a patient having some complaints about his floater. The angular size of the scan was 30°×30° corresponding to an 8 mm×8 mm retinal size.

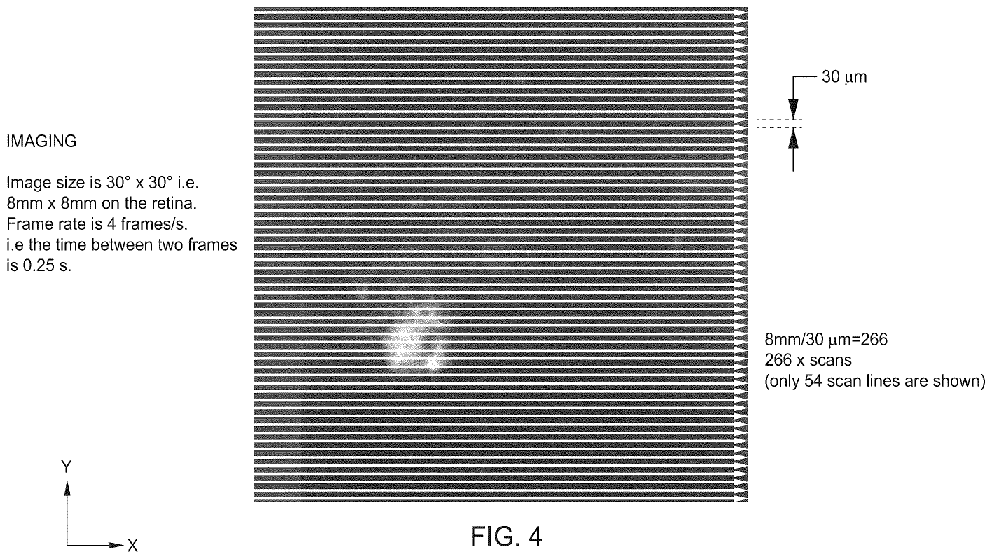

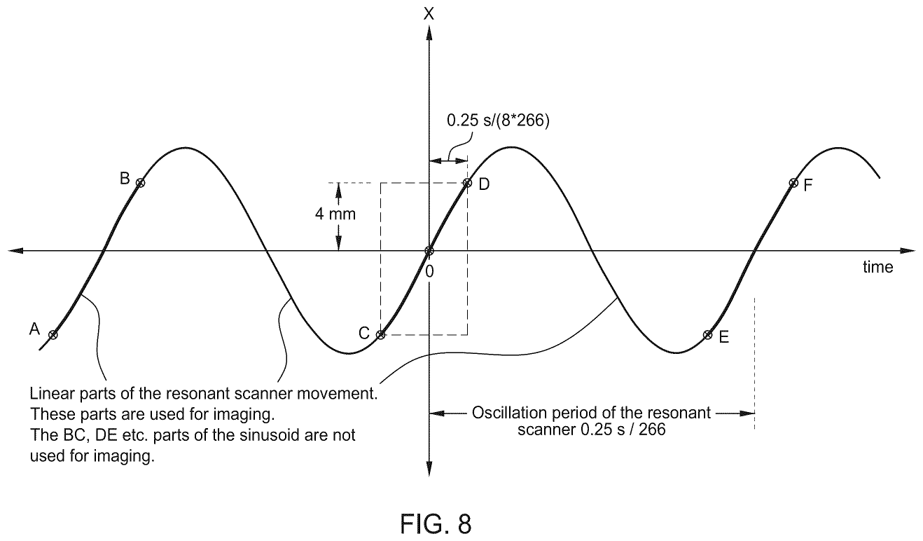

Referring now to FIG. 4 and FIG. 8, the horizontal lines in FIG. 4 illustrate the scanning pattern of the beam of the laser diode LD as scanned by the RX and RY scanning mirrors SM. The RX mirror is rotated by the resonant scanner. The angular deflection of the LD laser beam along the X direction is a sinusoidal function of the time, as shown in FIG. 8. To form the example image in FIG. 3, the segments AB, CD, and EF, etc., are used to scan the laser diode LD laser spot along the X direction from left to right in the segment of −4 mm to +4 m shown in FIG. 8. The segments AB, CD, and EF, etc., are about ¼ of the total oscillation period of the resonant scanner. In each of these segments, the speed of beam deflection can be regarded as being effectively constant. The segments BC, DE, etc., were used to move the beam along the Y direction with steps of 30 μm. Y direction steps of 30 μm result in a very good spatial resolution of the floater 114. To build one enface X/Y image, 8 mm/30 μm=266 horizontal scanning traces were used. To avoid crowding of the figure, only 54 horizontal scans are displayed. The output of the signal of the photodiode PD is ignored by the computing system 900 during the BC, DE, etc., periods. The frame rate of the video was 4 frames per second. Therefore, the oscillation period of the resonant scanner of the RX mirror is 0.25 s/266. The scanning time of the OD segment (FIG. 8) is about 0.25 s/(266*8) and the scanning distance is 4 mm. Thus, the scanning speed is 4 mm*266*8/0.25 s=34 m/s. The resonant scanner of the RX mirrors is very capable of achieving this scanning speed. The distance, angle, speed, and other values in the above example are exemplary only. Other values may also be achieved. In particular, the resonant scanner of the RX mirror and the galvo of the RY mirror may operate at slower or faster speeds than those described above.

The slope of the sinusoid in the OD segment may not be perfectly constant, which causes some image distortion along the X direction. Using the properties of the sinusoidal function, this distortion can be corrected prior to displaying the enface X/Y image. However, the accuracy of the laser treatment using the treatment laser TL is not affected by any distortion since the imaging and the treating laser beams are scanned with the same system 200.

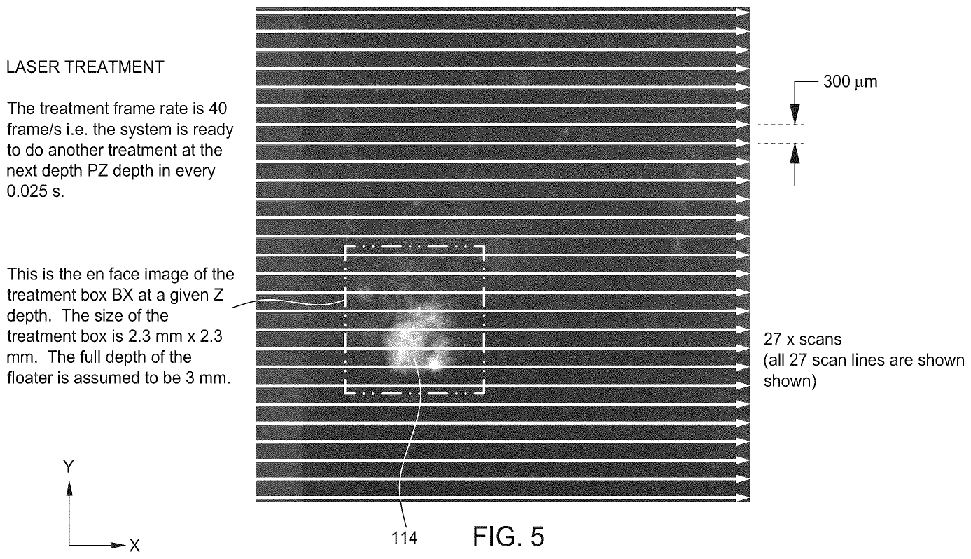

The scanning discussed above can be regarded as the design phase of the treatment procedure. In this phase, the x/y location and x/y size and shape of the floater 114 is determined for every PZ depth using an image-analyzing software known in the art. Combining the plurality of enface cross section images of the 3D image of the floater can be constructed and if needed can be displayed in a form of a video. In this example (FIGS. 4 and 5), an orthogonal treatment box is defined having an x/y size of 2.3 mm×2.3 mm and a depth of 3 mm. This treatment box BX is a closed surface fully enclosing the floater 114. In this example, the treatment box has a rectangular shape but the shape can have any closed 3D geometry corresponding to the actual 3D shape of the floater.

FIG. 5 shows an example scanning pattern to be used during treatment of the floater 114 with the treatment laser TL. The treatment laser TL beam and the laser diode LD beam are scanned with the same scanner, therefore the movement of the treatment laser spot is the same as the movement of the LD laser spot. During the treatment, the resonant frequency and the 34 m/s×scanning speed is unchanged but the Y line separation is increased from 30 μm to 300 μm. In this way, there will be not 266 but only 27 horizontal X scanning lines and the frame rate is increased from 4 frame per second to 40 frame per second. These values are exemplary only and these values may vary in correspondence with changes to the values used for imaging as described above with respect to FIG. 4.

For the treatment of the floater 114, 300 μm vertical line pitch was selected for the reason outlined below. Experiments conducted by the inventors have shown that floaters can be disintegrated with a plurality of 15 μJ laser pulses. The experiments also showed that 15 μJ laser pulses generate transient cavitation bubbles having 370 μm diameter. In this way, by filling the treatment box BX with laser pulses focused into a 300 μm×300 μm×300 μm matrix, the floater 114 can be fully disintegrated. To achieve a 300 μm laser spot separation along the X direction, the treatment laser TL may have a repetition rate substantially (e.g., within 1 Hz) equal to the scanning speed divided by the laser spot separation, i.e., (34 m/s)/300 μm=113 kHz. The typical repetition rate of CPA lasers is up to about 2 MHz. However, using acoustooptical deflectors or Pockels cells, the pulses can be selected for transmission into the vitreous 110 at the required repetition rate. To have 300 μm laser spot separation in the Z direction, the depth focusing lens actuator LA should be activated between two layers as described above. In experimentation using system 200, 300 μm Z steps was achieved by increasing the focusing power of the treatment laser TL beam by about 0.8 diopter. In some embodiments, laser treatment of the floater begins at the deepest Z layer (closest to the retina 112) and move the treatment stepwise in the anterior direction (toward the cornea 102). In this case, the longer living cavitation bubbles do not block passage of the treatment laser TL beam from reaching the deeper lying parts of the floater 114. Another advantage of starting at the deepest Z layer is that the longer living bubbles partially protect the retina from exposure to the treatment laser TL beam.

During the treatment of the floater 114, part of the treatment laser TL energy reaches the retina 112. To avoid retinal damage the exposure should be kept below the so-called American National Standards Institute (ANSI) Maximum Permissible Exposure (MPE) limits, such as those described in ANSI Z136.1-2014, which is hereby incorporated herein by reference in its entirety. The ANSI MPE limit depends on the laser pulse energy, laser repetition rate, numerical aperture of the focused laser beam, number of laser pulses used, the distance in the PZ direction from the retina 112, the laser wavelength, laser pulse duration, the scanning pattern, and other parameters.

The system 200 may be used to image the floater even during the treatment procedure. However, the spatial resolution of the image in the vertical (i.e., Y) direction is decreased from 30 μm to 300 μm vertical pitch size in the examples described above. The 300 μm vertical resolution is enough to track the possible intra-treatment slow movement of the floater 114 and to facilitate possible re-aiming at the floater with the treatment laser.

In the following description, the closed “treatment box BX” is used to identify the boundary with the understanding that other boundaries may be used in a like manner. For example, the boundary may be more precisely defined in the form of an oriented box that has a rectangular shape with sides that are not necessarily parallel with the X and Y axis. The boundary may also be a non-rectangular shape tracing an estimated boundary of the portion of the image corresponding to a floater 114. For example, the perimeter of a blob of pixels in each X/Y image having an intensity above a threshold may be used as the boundary.

After determining the location, the shape and the dimensions of the treatment box BX, the vitreous 110 may be scanned and treated using the focused treatment laser TL beam. As already described above, in some embodiments, the vertical scanning pitch in the Y direction for the treatment laser TL may be larger than the scanning pitch for the laser diode LD during the imaging phase. For example, the scanning pitch may be between 5 and 15 times, such as 10 times, greater than the scanning pitch for the laser diode LD. For example, in the illustrated example, the vertical scanning pitch is 300 μm. When scanning with the treatment laser TL, the increased vertical scanning pitch may be achieved by increasing the scanning steps during the BC,DE, etc., periods (FIG. 8.) in the Y direction, e.g., 10 times greater to increase the vertical scanning pitch from 30 μm to 300 μm. As described above, the vertical scanning pitch may be selected based on the diameter of the cavitation bubbles.

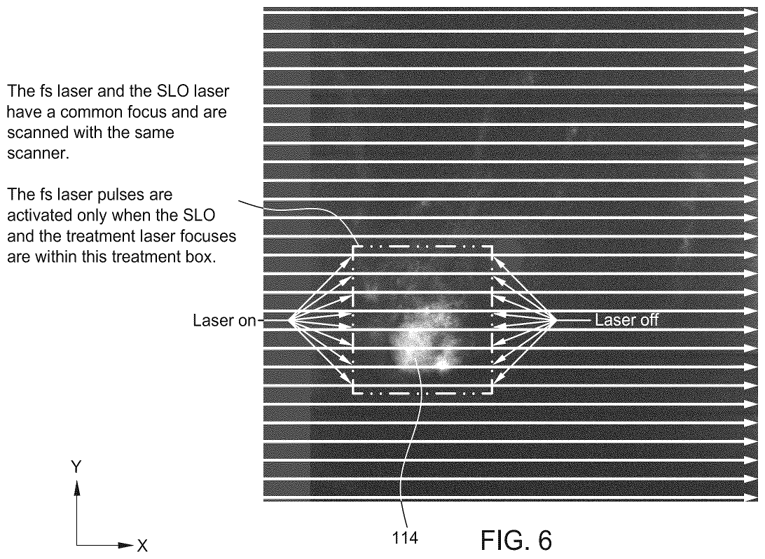

Referring to FIG. 6, during treatment, the treatment laser TL is scanned across the vitreous 110 in three dimensions. For each depth position PZ, the treatment laser TL is turned off for all positions PXY outside the treatment box BX obtained from the X/Y image for the current depth position PZ. For positions PXY inside the bounding box BX, the treatment laser TL is turned on and pulses from the treatment laser TL are selectively permitted to reach the vitreous 110 in order to destroy the floater 114. The laser “on” and the laser “off” transition points are indicated with arrows in FIG. 6.

In cases where several floaters 114 are detected during the imaging phase, a plurality of treatment boxes BX can be defined. The plurality of floaters 114 can be treated either one-by-one or all at once starting with the deepest floater. In this way the long living cavitation bubbles do not cast a shadow onto the deeper lying floaters.

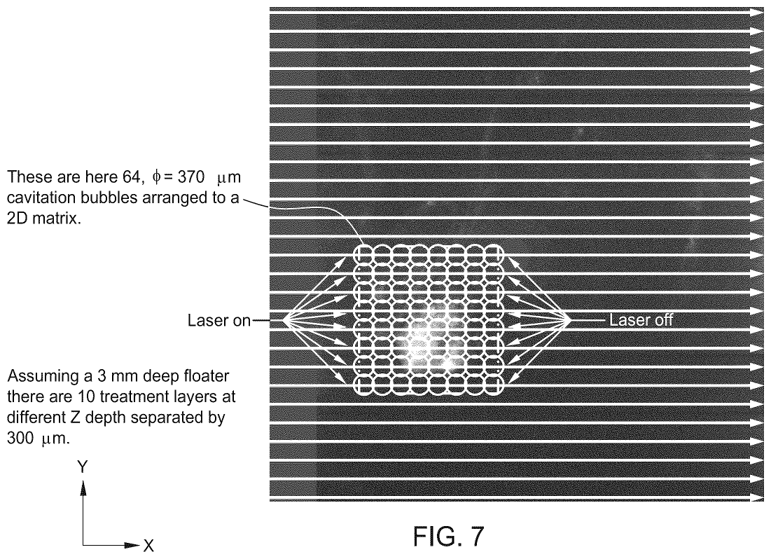

Referring to FIG. 7, a pulse from the treatment laser TL incident on a point PXY, PZ within the vitreous will create a momentary cavitation bubble that will disintegrate a portion of the floater located in the near vicinity of point PXY, PZ. As described above, the horizontal pulse pitch at which pulses are allowed to reach the vitreous 110 may be selected based on the estimated diameter for the cavitation bubbles. The vertical scanning pitch along the Y direction and the depth scanning pitch along the Z direction may likewise be selected based on the estimated diameter of the cavitation bubbles. For example, the horizontal pulse pitch, vertical scanning pitch, and depth scanning pitch may be selected to be between 0.5 and 1.5, between 0.7 and 1.3, or between 0.9 and 1.1 times the estimated cavitation bubble diameter. For example, the horizontal pulse pitch may be selected to be between 0.1 and 0.4 mm. The previous paragraph describes the algorithm of selecting the treatment parameters. These parameters depend on the volume and the shape of the floater 114 and the energy of the laser pulse to be used, which determines the size of the cavitation bubbles.

A degree of synchronization is required between the scanning by the scanning mirror SM and the activation and pulse selection of the treatment laser TL. Assuming a 2 MHz regenerative amplifier, the spatial accuracy of the treatment laser TL is (34 m/s)/(2 MHz)=17 μm. This spatial accuracy is completely satisfactory since the spatial 17 μm accuracy is only 4.6% of the size of the cavitation bubble diameter (370 μm).

The above-described parameters and calculations are exemplary only. The parameters depend on the size and shape of the floaters 114 and on the laser pulse energy, the resonant frequency of the scanner etc. The following are ranges of possible parameters for the treatment laser TL and laser diode LD: the treatment laser TL can have a pulse duration 10 ps to 50 fs; the repetition rate of the treatment laser can be between 1 kHz and 2 MHz; the wavelength of the treatment laser TL can be 650 nm to 2 μm; the treatment laser TL pulse energy on the target may be from 1 to 50 μJ, from 5 to 25 μJ, or from 10 to 20 μJ; the spatial separation of the laser treatment spots along the X scanning direction may be from 10 μm to 1 mm; the vertical scanning pitch during imaging with the laser diode LD may be from 5 μm to 200 μm; the vertical scanning pitch for the treatment laser TL may be from 30 μm to 1 mm; the depth scanning pitch for the treatment laser TL may be from 30 μm to 1 mm; and the frame rate of during imaging with the laser diode LD may be from 0.5 to 25 frame/s.

Various refinements may be implemented to improve the precision of the approach described above. Floaters may be motile and will move in response to saccadic movement of the eye 100. Accordingly, a patient may be instructed to stare at a fixation target for at least two seconds prior to and during imaging and treatment in order to reduce change in position of a floater 114 between imaging and treatment and during treatment.

As the name “floater” indicates, floaters 114 can spatially move. Upon fixation of the gaze this movement is rather slow, about 0.02° per second to 0.10 per second. Remembering that the system 200 is capable to image the floater 114 even during the laser treatment, the system 200 can be programmed to always track this movement and reposition the treatment box BX during treatment in correspondence with the tracked movement.

As a floater 114 is disintegrated, the expansion of the cavitation bubbles may nudge the floater 114. Likewise, the buoyancy forces of the cavitation bubbles may shift the floater 114. The system 200 can be programmed to track these movements during treatment and properly reposition the treatment box BX during treatment in response to this movement as well.

Upon fixation of the gaze, there is a small, fast, and spatially random movement of the eye called microsaccades. The purpose of the microsaccades is to avoid the fading of the image. The amplitude of the microsaccades is about 0.8°, the duration is about 0.012 second, the frequency is about 0.9 per second, and the angular speed can reach 40° per second. To track these fast and short movements with an eye tracker and move the treatment box BX would be very technically challenging. Therefore, the treatment box BX may be made larger than the actual size of the floater 114 by about 0.5 mm in all of the X, Y, and Z directions.

To increase the speed of floater removal, beam multiplexing can be used. Beam multiplexing means that the single beam of the treatment laser TL beam is optically modified to have not only one but simultaneously a plurality of focused laser spots. Multiplexing can be achieved in the X/Y plane or in the Z direction or in a combination of these. Multiplexing can be achieved by optical elements incorporated into the system 200, such as diffractive optical elements, spatial phase modulators, birefringent optical components, or different kinds of interferometers. These optical elements may be positioned between the scanning mirror SM and the lens actuator LA. For example, each beam emitted by these elements may be focused by a different set of lenses and corresponding lens actuators.

FIG. 9 illustrates an example computing system 900 that implements, at least partly, one or more functionalities described herein with respect to FIGS. 1 to 8. The computing system 900 may be integrated with an imaging device, such as the system 200, or be a separate computing device receiving images of a patient's eye from the imaging device.

As shown, computing system 900 includes a central processing unit (CPU) 902, one or more I/O device interfaces 904, which may allow for the connection of various I/O devices 914 (e.g., keyboards, displays, mouse devices, pen input, etc.) to computing system 900, network interface 906 through which computing system 900 is connected to network 990, a memory 908, storage 910, and an interconnect 912.

In cases where computing system 900 is an imaging system, such the system 200, the computing system 900 may further include one or more optical components for obtaining ophthalmic imaging of a patient's eye as well as any other components known to one of ordinary skill in the art.

CPU 902 may retrieve and execute programming instructions stored in the memory 908. Similarly, CPU 902 may retrieve and store application data residing in the memory 908. The interconnect 912 transmits programming instructions and application data, among CPU 902, I/O device interface 904, network interface 906, memory 908, and storage 910. CPU 902 is included to be representative of a single CPU, multiple CPUs, a single CPU having multiple processing cores, and the like.

Memory 908 is representative of a volatile memory, such as a random access memory, and/or a nonvolatile memory, such as nonvolatile random access memory, phase change random access memory, or the like. As shown, memory 908 may store a scanning module 916 configured to cause the system 200 to image the vitreous 110 of a patient's eye as described above. The memory 908 may further sore a treatment module 918 configured to control the system 200 to destroy floaters as described above.

Storage 910 may be non-volatile memory, such as a disk drive, solid state drive, or a collection of storage devices distributed across multiple storage systems. Storage 910 may optionally store the X/Y images 920 captured using the system 200 for subsequent processing to identify the boundaries of floaters as described above.

### ADDITIONAL CONSIDERATIONS

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method comprising:
focusing a first light from a first laser at a plurality of points within a vitreous of a patient's eye using a scanner system;
simultaneously measuring a reflected light from the plurality of points while focusing the first light at the plurality of points;
determining, by a computer system, that the reflected light from a portion of the plurality of points corresponds to one or more vitreous floaters; and
in response to determining that the reflected light from the portion of the plurality of points corresponds to the one or more vitreous floaters, focusing a second light from a second laser at the portion of the plurality of points using the scanner system in order to disintegrate the one or more vitreous floaters.

2. The method of claim 1, wherein the first laser is an infrared laser diode.

3. The method of claim 1, wherein the second laser is a pulsed laser.

4. The method of claim 3, wherein the second laser produces pulses having pulse energies of between 1 μJ and 50 μJ.

5. The method of claim 3, wherein the second laser produces pulses having pulse energies of between 10 μJ and 20 μJ.

6. The method of claim 3, wherein the second laser produces pulses having a duration of between 10 ps and 50 fs.

7. The method of claim 3, wherein the second laser has a wavelength of between 650 nm and 2 μm.

8. The method of claim 1, wherein the scanner system includes a resonant scanner.

9. The method of claim 1, wherein the scanner system comprises optical elements configured to simultaneously focus the second light at the portion of the plurality of points, wherein the optical elements comprise one or more of diffractive optical elements, one or more spatial phase modulators, or one or more interferometers.

10. The method of claim 9, further comprising adjusting one or more adjustable lenses to change a depth of a focal point of the first light and a focal point of the second light within the vitreous of the patient's eye.

11. A system comprising:
a three-dimensional scanner;
a first laser for imaging vitreous floaters;
a second laser configured to emit pulses sufficient to disintegrate the vitreous floaters; and
one or more combining optics configured to direct a first light from the first laser and a second light from the second laser into the three-dimensional scanner, the one or more combining optics configured to place a second focal point of the second laser within 0.01 μm of a second focal point of the second laser.

12. The system of claim 11, wherein the three-dimensional scanner comprises a mirror driven by a resonant scanner.

13. The system of claim 12, wherein the three-dimensional scanner comprises one or more adjustable lenses.

14. The system of claim 13, wherein the one or more adjustable lenses comprise one or more lenses mounted to an actuator.

15. The system of claim 13, wherein the one or more adjustable lenses comprises an electrically tunable optofluidic lens.

16. The system of claim 12, wherein the one or more combining optics comprise one or more beam splitters positioned to receive the first light from the first laser and the second light from the second laser.

17. The system of claim 16, wherein the one or more beam splitters comprise:
a first beam splitter configured to pass a first portion of the first light and direct a second portion of the second light to be parallel to the first portion.

18. The system of claim 17, wherein the one or more beam splitters comprise:
a second beam splitter positioned between the first beam splitter and the first laser, the second beam splitter configured to direct a third portion of the first light reflected from the vitreous floaters onto a photodiode.

19. The system of claim 18, further comprising a confocal pinhole filter positioned between the second beam splitter and the photodiode.

20. The system of claim 19, further comprising a lens positioned between the second beam splitter and the confocal pinhole filter such that a focal point of the third portion of the first light is positioned at the confocal pinhole filter.
